(19) United States
(12) Patent Application Publication
(10) Pub. No.: US 2025/0377229 A1
(43) Pub. Date: Dec. 11, 2025

(54) System, Method, and Computer Program Product for Inhibiting Spurious Flow Measurement by Ultrasonic Flow Sensors

(71) Applicant: Becton, Dickinson and Company, Franklin Lakes, NJ (US)

(72) Inventors: Phillip Fiorenzo, North Haledon, NJ (US); Kaushal Verma, Bridgewater, NJ (US)

(21) Appl. No.: 18/735,932

(22) Filed: Jun. 6, 2024

(51) Int. Cl. G01F 1/66; G01F 1/667

(52) U.S. Cl. CPC G01F 1/662; G01F 1/667

(57) ABSTRACT

Systems, methods, and computer program products are provided for inhibiting spurious flow measurement by ultrasonic flow sensors. An example system includes an ultrasonic flow sensor that includes a flow tube for delivering a fluid from a fluid source, a first piezoelectric sensor or transducer, and a second piezoelectric sensor or transducer; and at least one processor configured to: receive a first time-series generated by the ultrasonic flow; receive a second time-series generated by the ultrasonic flow sensor; and determine, based on the first time-series and the second time-series, whether to modify a previous amount associated with at least one attribute of the fluid with an interval amount associated with the at least one attribute of the fluid.

### BACKGROUND

### Technical Field

This disclosure relates generally to ultrasonic flow sensors and, in some non-limiting embodiments or aspects, to systems, methods, and computer program products for inhibiting spurious flow measurement by ultrasonic flow sensors.

### Technical Considerations

Ultrasonic flow sensors may be used measure a flow rate and a volume of a fluid (e.g., a medication, etc.) delivered from a medication container (e.g., a syringe, etc.) to a patient. However, existing ultrasonic flow sensors do not have a mechanism for inhibiting or preventing output of spurious flow measurement (e.g., volume accumulation when there is no actuation of the syringe that delivers the fluid, etc.) For example, a presence of an air bubble in a fluid flow path of an ultrasonic flow sensor may alter a sensor signal, which when analyzed can lead to spurious or fake flow measurements.

### SUMMARY

Accordingly, provided are improved systems, methods, and computer program products for inhibiting spurious flow measurement by ultrasonic flow sensors.

According to non-limiting embodiments or aspects, provided is a system including: an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube; and at least one processor configured to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

In some non-limiting embodiments or aspects, the at least one attribute of the fluid includes at least one of the following attributes of the fluid: a flow rate, a volume, or any combination thereof.

In some non-limiting embodiments or aspects, the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

In some non-limiting embodiments or aspects, the at least one processor is configured to determine, based on the first time-series and the second time-series, whether to modify the previous amount associated with at least one attribute of the fluid with the interval amount associated with the at least one attribute of the fluid by: calculating, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and determining whether the average amplitude satisfies a threshold amplitude.

In some non-limiting embodiments or aspects, the at least one processor is configured to determine, based on the first time-series and the second time-series, whether to modify the previous amount associated with at least one attribute of the fluid with the interval amount associated with the at least one attribute of the fluid by: calculating, based on the first time series and the second time series, a relative difference between the first time series and the second time series; and determining whether the relative difference between the first time series and the second time series satisfies a threshold difference.

In some non-limiting embodiments or aspects, the at least one processor is configured to determine, based on the first time-series and the second time-series, whether to modify the previous amount associated with at least one attribute of the fluid with the interval amount associated with the at least one attribute of the fluid by: calculating a standard deviation of a flow rate of the fluid in the flow tube over a plurality of time intervals before the time interval; and determining whether the standard deviation of the flow rate of the fluid in the flow tube satisfies a threshold deviation.

In some non-limiting embodiments or aspects, the at least one processor is configured to determine, based on the first time-series and the second time-series, whether to modify the previous amount associated with at least one attribute of the fluid with the interval amount associated with the at least one attribute of the fluid by: determining, based on the first time series and the second time series, a plurality of differences in transit time between the at least one first ultrasonic signal and the at least one second ultrasonic signal; calculating an average difference in transit time of the plurality of differences in transit time; and determining whether a range of the differences in transit time between the at least one first ultrasonic signal and the at least one second ultrasonic signal satisfies a threshold range determined based on the average difference in transit time of the plurality of differences in transit time.

In some non-limiting embodiments or aspects, the at least one processor is configured to determine, based on the first time-series and the second time-series, whether to modify the previous amount associated with at least one attribute of the fluid with the interval amount associated with the at least one attribute of the fluid by: providing, as input to at least one machine learning model, the first time-series and the second time-series; and receiving, as output from the at least one machine learning model, a prediction of whether to modify the previous amount associated with the at least one attribute of the fluid with the interval amount associated with the at least one attribute.

According to non-limiting embodiments or aspects, provided is an ultrasonic flow sensor including: a flow tube for delivering a fluid from a fluid source; a first piezoelectric sensor or transducer arranged at an upstream position of the flow tube; a second piezoelectric sensor or transducer arranged at a downstream position of the flow tube; and at least one processor configured to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

In some non-limiting embodiments or aspects, the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

According to non-limiting embodiments or aspects, provided is a method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube, the method including: receiving, with at least one processor, a first time-series generated by the ultrasonic flow sensor by causing the first piezoelectric sensor or transducer to transmit at least one first ultrasonic signal to the second piezoelectric sensor or transducer; receiving, with the at least one processor, a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determining, with the at least one processor, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

In some non-limiting embodiments or aspects, the at least one attribute of the fluid includes at least one of the following attributes of the fluid: a flow rate, a volume, or any combination thereof.

In some non-limiting embodiments or aspects, the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

In some non-limiting embodiments or aspects, determining, with the at least one processor, based on the first time-series and the second time-series, whether to modify the previous amount associated with at least one attribute of the fluid with the interval amount associated with the at least one attribute of the fluid includes: calculating, with the at least one processor, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and determining, with the at least one processor, whether the average amplitude satisfies a threshold amplitude.

In some non-limiting embodiments or aspects, determining, with the at least one processor, based on the first time-series and the second time-series, whether to modify the previous amount associated with at least one attribute of the fluid with the interval amount associated with the at least one attribute of the fluid includes: calculating, with the at least one processor, based on the first time series and the second time series, a relative difference between the first time series and the second time series; and determining, with the at least one processor, whether the relative difference between the first time series and the second time series satisfies a threshold difference.

In some non-limiting embodiments or aspects, determining, with the at least one processor, based on the first time-series and the second time-series, whether to modify the previous amount associated with at least one attribute of the fluid with the interval amount associated with the at least one attribute of the fluid includes: calculating, with the at least one processor, a standard deviation of a flow rate of the fluid in the flow tube over a plurality of time intervals before the time interval; and determining, with the at least one processor, whether the standard deviation of the flow rate of the fluid in the flow tube satisfies a threshold deviation.

In some non-limiting embodiments or aspects, determining, with the at least one processor, based on the first time-series and the second time-series, whether to modify the previous amount associated with at least one attribute of the fluid with the interval amount associated with the at least one attribute of the fluid includes: determining, with the at least one processor, based on the first time series and the second time series, a plurality of differences in transit time between the at least one first ultrasonic signal and the at least one second ultrasonic signal; calculating, with the at least one processor, an average difference in transit time of the plurality of differences in transit time; and determining, with the at least one processor, whether a range of the differences in transit time between the at least one first ultrasonic signal and the at least one second ultrasonic signal satisfies a threshold range determined based on the average difference in transit time of the plurality of differences in transit time.

In some non-limiting embodiments or aspects, determining, with the at least one processor, based on the first time-series and the second time-series, whether to modify the previous amount associated with at least one attribute of the fluid with the interval amount associated with the at least one attribute of the fluid includes: providing, with the at least one processor, as input to at least one machine learning model, the first time-series and the second time-series; and receiving, with the at least one processor, as output from the at least one machine learning model, a prediction of whether to modify the previous amount associated with the at least one attribute of the fluid with the interval amount associated with the at least one attribute.

According to non-limiting embodiments or aspects, provided is a computer program product including a non-transitory computer readable medium including program instructions method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube which, when executed by at least one processor, cause the at least one processor to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

In some non-limiting embodiments or aspects, the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

Further non-limiting embodiments or aspects are set forth in the following numbered clauses:

Clause 1. A system comprising: an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube; and at least one processor configured to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

Clause 2. The system of clause 1, wherein the at least one attribute of the fluid includes at least one of the following attributes of the fluid: a flow rate, a volume, or any combination thereof.

Clause 3. The system of clause 1 or clause 2, wherein the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

Clause 4. The system of any of clauses 1-3, wherein the at least one processor is configured to determine, based on the first time-series and the second time-series, whether to modify the previous amount associated with at least one attribute of the fluid with the interval amount associated with the at least one attribute of the fluid by: calculating, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and determining whether the average amplitude satisfies a threshold amplitude.

Clause 5. The system of any of clauses 1-4, wherein the at least one processor is configured to determine, based on the first time-series and the second time-series, whether to modify the previous amount associated with at least one attribute of the fluid with the interval amount associated with the at least one attribute of the fluid by: calculating, based on the first time series and the second time series, a relative difference between the first time series and the second time series; and determining whether the relative difference between the first time series and the second time series satisfies a threshold difference.

Clause 6. The system of any of clauses 1-5, wherein the at least one processor is configured to determine, based on the first time-series and the second time-series, whether to modify the previous amount associated with at least one attribute of the fluid with the interval amount associated with the at least one attribute of the fluid by: calculating a standard deviation of a flow rate of the fluid in the flow tube over a plurality of time intervals before the time interval; and determining whether the standard deviation of the flow rate of the fluid in the flow tube satisfies a threshold deviation.

Clause 7. The system of any of clauses 1-6, wherein the at least one processor is configured to determine, based on the first time-series and the second time-series, whether to modify the previous amount associated with at least one attribute of the fluid with the interval amount associated with the at least one attribute of the fluid by: determining, based on the first time series and the second time series, a plurality of differences in transit time between the at least one first ultrasonic signal and the at least one second ultrasonic signal; calculating an average difference in transit time of the plurality of differences in transit time; and determining whether a range of the differences in transit time between the at least one first ultrasonic signal and the at least one second ultrasonic signal satisfies a threshold range determined based on the average difference in transit time of the plurality of differences in transit time.

Clause 8. The system of any of clauses 1-7, wherein the at least one processor is configured to determine, based on the first time-series and the second time-series, whether to modify the previous amount associated with at least one attribute of the fluid with the interval amount associated with the at least one attribute of the fluid by: providing, as input to at least one machine learning model, the first time-series and the second time-series; and receiving, as output from the at least one machine learning model, a prediction of whether to modify the previous amount associated with the at least one attribute of the fluid with the interval amount associated with the at least one attribute.

Clause 9. An ultrasonic flow sensor comprising: a flow tube for delivering a fluid from a fluid source; a first piezoelectric sensor or transducer arranged at an upstream position of the flow tube; a second piezoelectric sensor or transducer arranged at a downstream position of the flow tube; and at least one processor configured to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

Clause 10. The ultrasonic flow sensor of clause 9, wherein the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

Clause 11. A method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube, the method comprising: receiving, with at least one processor, a first time-series generated by the ultrasonic flow sensor by causing the first piezoelectric sensor or transducer to transmit at least one first ultrasonic signal to the second piezoelectric sensor or transducer; receiving, with the at least one processor, a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determining, with the at least one processor, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

Clause 12. The method of clause 11, wherein the at least one attribute of the fluid includes at least one of the following attributes of the fluid: a flow rate, a volume, or any combination thereof.

Clause 13. The method of clause 11 or clause 12, wherein the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

Clause 14. The method of any of clauses 11-13, wherein determining, with the at least one processor, based on the first time-series and the second time-series, whether to modify the previous amount associated with at least one attribute of the fluid with the interval amount associated with the at least one attribute of the fluid includes: calculating, with the at least one processor, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and determining, with the at least one processor, whether the average amplitude satisfies a threshold amplitude.

Clause 15. The method of any of clauses 11-14, wherein determining, with the at least one processor, based on the first time-series and the second time-series, whether to modify the previous amount associated with at least one attribute of the fluid with the interval amount associated with the at least one attribute of the fluid includes: calculating, with the at least one processor, based on the first time series and the second time series, a relative difference between the first time series and the second time series; and determining, with the at least one processor, whether the relative difference between the first time series and the second time series satisfies a threshold difference.

Clause 16. The method of any of clauses 11-15, wherein determining, with the at least one processor, based on the first time-series and the second time-series, whether to modify the previous amount associated with at least one attribute of the fluid with the interval amount associated with the at least one attribute of the fluid includes: calculating, with the at least one processor, a standard deviation of a flow rate of the fluid in the flow tube over a plurality of time intervals before the time interval; and determining, with the at least one processor, whether the standard deviation of the flow rate of the fluid in the flow tube satisfies a threshold deviation.

Clause 17. The method of any of clauses 11-16, wherein determining, with the at least one processor, based on the first time-series and the second time-series, whether to modify the previous amount associated with at least one attribute of the fluid with the interval amount associated with the at least one attribute of the fluid includes: determining, with the at least one processor, based on the first time series and the second time series, a plurality of differences in transit time between the at least one first ultrasonic signal and the at least one second ultrasonic signal; calculating, with the at least one processor, an average difference in transit time of the plurality of differences in transit time; and determining, with the at least one processor, whether a range of the differences in transit time between the at least one first ultrasonic signal and the at least one second ultrasonic signal satisfies a threshold range determined based on the average difference in transit time of the plurality of differences in transit time.

Clause 18. The system of any of clauses 11-17, wherein determining, with the at least one processor, based on the first time-series and the second time-series, whether to modify the previous amount associated with at least one attribute of the fluid with the interval amount associated with the at least one attribute of the fluid includes: providing, with the at least one processor, as input to at least one machine learning model, the first time-series and the second time-series; and receiving, with the at least one processor, as output from the at least one machine learning model, a prediction of whether to modify the previous amount associated with the at least one attribute of the fluid with the interval amount associated with the at least one attribute.

Clause 19. A computer program product including a non-transitory computer readable medium including program instructions method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube which, when executed by at least one processor, cause the at least one processor to: receive a first time-series generated by the ultrasonic flow sensor by causing the first piezoelectric sensor or transducer to transmit at least one first ultrasonic signal to the second piezoelectric sensor or transducer; receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

Clause 20. The computer program product of clause 19, wherein the first time series includes a plurality of first amplitudes of the at least one first ultrasonic signal received at the second piezoelectric sensor or transducer at a plurality of first time points in the time interval, wherein the second time series includes a plurality of second amplitudes of the at least one second ultrasonic signal received at the first piezoelectric sensor or transducer at a plurality of second time points in the time interval, and wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosed subject matter.

### BRIEF DESCRIPTION OF THE DRAWINGS

Additional advantages and details are explained in greater detail below with reference to the non-limiting, exemplary embodiments that are illustrated in the accompanying schematic figures, in which:

FIG. 1A is a schematic diagram of a system for inhibiting spurious flow measurement by ultrasonic flow sensors, according to some non-limiting embodiments or aspects;

FIG. 1B is a perspective view of example components of a flow sensor system of the system for inhibiting spurious flow measurement by ultrasonic flow sensors of FIG. 1A, according to some non-limiting embodiments or aspects;

FIG. 1C is a cross-sectional view of example components of an ultrasonic flow sensor of a flow sensor system of the system for inhibiting spurious flow measurement by ultrasonic flow sensors of FIG. 1A, according to some non-limiting embodiments or aspects;

FIG. 2 is a schematic diagram of example components of one or more devices or systems of FIG. 1A, according to some non-limiting embodiments or aspects;

FIG. 3 is a flow diagram of a method for inhibiting spurious flow measurement by ultrasonic flow sensors, according to some non-limiting embodiments or aspects;

FIG. 4 is a flow diagram of a method for inhibiting spurious flow measurement by ultrasonic flow sensors, according to some non-limiting embodiments or aspects;

FIG. 5 is a flow diagram of a method for inhibiting spurious flow measurement by ultrasonic flow sensors, according to some non-limiting embodiments or aspects; and

FIG. 6 is a graph of example “up” and “down” time series signals showing offset due to fluid flow.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the embodiments as they are oriented in the drawing figures. However, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosed subject matter. Hence, specific dimensions and other physical characteristics related to the embodiments or aspects disclosed herein are not to be considered as limiting.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. In addition, reference to an action being “based on” a condition may refer to the action being “in response to” the condition. For example, the phrases “based on” and “in response to” may, in some non-limiting embodiments or aspects, refer to a condition for automatically triggering an action (e.g., a specific operation of an electronic device, such as a computing device, a processor, and/or the like).

As used herein, the term “communication” may refer to the reception, receipt, transmission, transfer, provision, and/or the like of data (e.g., information, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit processes information received from the first unit and communicates the processed information to the second unit. In some non-limiting embodiments or aspects, a message may refer to a network packet (e.g., a data packet and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer or other form of non-mobile computer.

As used herein, the term “server” may refer to or include one or more computing devices that are operated by or facilitate communication and processing for multiple parties in a network environment, such as the Internet, although it will be appreciated that communication may be facilitated over one or more public or private network environments and that various other arrangements are possible. Further, multiple computing devices (e.g., servers, point-of-sale (POS) devices, mobile devices, etc.) directly or indirectly communicating in the network environment may constitute a “system.”

As used herein, the term “system” may refer to one or more computing devices or combinations of computing devices (e.g., processors, servers, client devices, software applications, components of such, and/or the like). Reference to “a device,” “a server,” “a processor,” and/or the like, as used herein, may refer to a previously-recited device, server, or processor that is recited as performing a previous step or function, a different device, server, or processor, and/or a combination of devices, servers, and/or processors. For example, as used in the specification and the claims, a first device, a first server, or a first processor that is recited as performing a first step or a first function may refer to the same or different device, server, or processor recited as performing a second step or a second function.

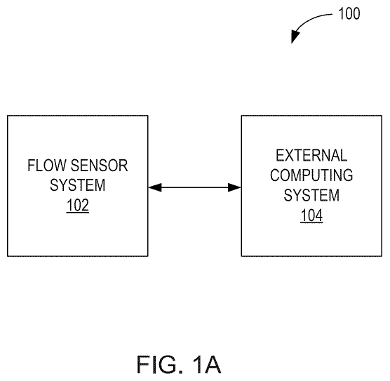

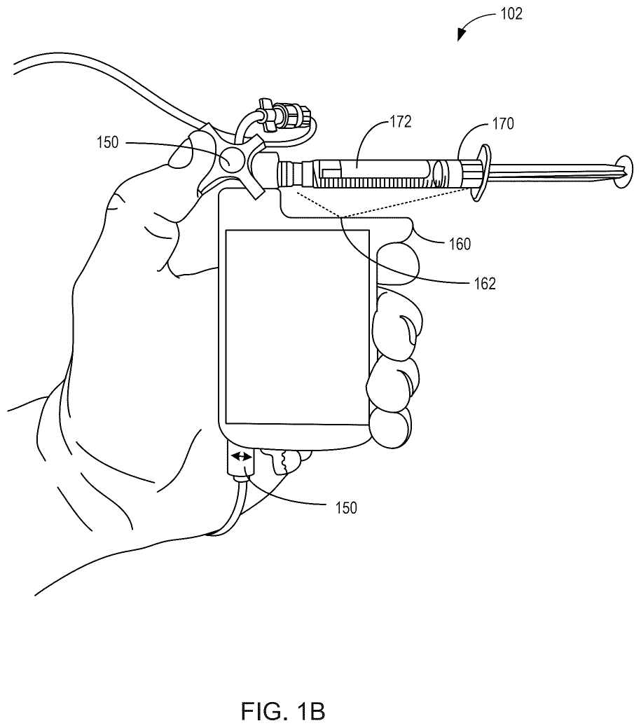

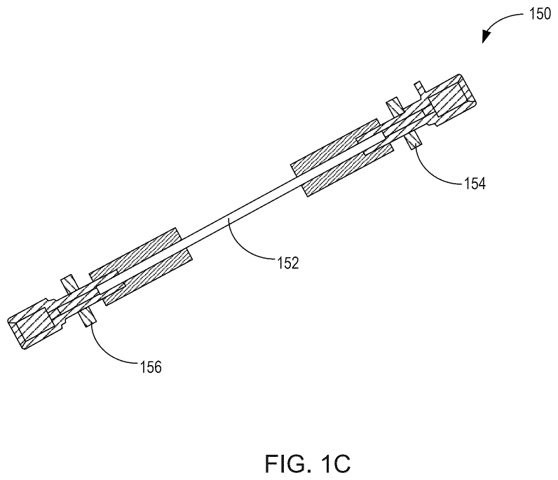

Referring now to FIG. 1A, shown is a schematic diagram of a system for calibrating ultrasonic flow sensors, according to some non-limiting embodiments or aspects. As shown in FIG. 1A, system 100 may include flow sensor system 102 and/or external computing system 104. Systems and/or devices of system 100 can interconnect via wired connections, wireless connections, or a combination of wired and wireless connections.

Flow sensor system 102 may include one or more devices capable of receiving information and/or data from external computing device 104 and/or communicating information and/or data to external computing device 104. For example, flow sensor system 102 may include one or more computing systems including one or more processors (e.g., one or more computing devices, one or more mobile computing devices, one or more digital signal processors (DSPs), etc.). In some non-limiting embodiments or aspects, flow sensor system 102 may include the flow sensor system in U.S. Patent Application Publication No. 2021/0231471 or U.S. Pat. No. 10,072,959.

Referring also to FIG. 1B, FIG. 1B is a perspective view of example components of a flow sensor system of the system for calibrating ultrasonic flow sensors of FIG. 1A, according to some non-limiting embodiments or aspects. As shown in FIG. 1B, flow sensor system 102 may include ultrasonic flow sensor 150 and/or base 160. For example, ultrasonic flow sensor 150 may be configured to be removably, physically, and/or electrically connected to base 160. Syringe 170 may be configured to physically connect to flow sensor 160 (e.g., via a fluid injection port, etc.). Syringe 170 may include tag or label 172, which may include an NFC tag (e.g., an RFID tag, etc.) embedded in tag or label 104, a barcode, a QR code, an AprilTag, or the like. Base 160 may include at least one sensor 162 configured to read and/or decode medication information from tag or label 172 on syringe 170. The medication information may include at least one expected medication type associated with at least one medication contained in syringe 170, such as a medication identifier (e.g., a unique medication identifier associated with the medication contained in syringe 170, etc.). For example, the at least one sensor 162 of base 160 may include one or more computing devices, chips, contactless transmitters, contactless transceivers, NFC transmitters/receivers, RFID transmitters/receivers, contact based transmitters/receivers, optical sensors or scanners, barcode readers, or the like that are configured to read and/or decode the medication information stored or encapsulated in tag or label 172.

Referring also to FIG. 1C, FIG. 1C is a cross-sectional view of components of an ultrasonic flow sensor of a flow sensor system of the system for calibrating ultrasonic flow sensors of FIG. 1A, according to some non-limiting embodiments or aspects. As shown in FIG. 1C, ultrasonic flow sensor 150 may include flow tube 152 that defines a fluid flow path of ultrasonic flow sensor 150, first piezoelectric sensor or transducer 154 arranged at an upstream position of flow tube 152, and/or second piezoelectric sensor or transducer 156 arranged at a downstream position of flow tube 152.

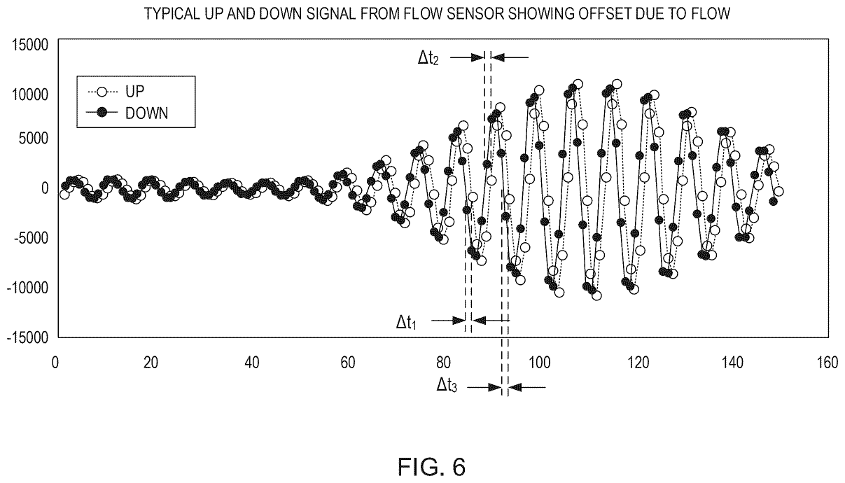

Ultrasonic flow sensor 150 may be configured to generate a time-series corresponding to (e.g., corresponding to, representative of, quantifying, measuring, etc.) a flow of at least one fluid through a fluid flow path of ultrasonic flow sensor 150. For example, first piezoelectric sensor or transducer 154 and second piezoelectric sensor or transducer 156 may be configured to generate the time-series corresponding to the flow of the at least one fluid through the fluid flow path of ultrasonic flow sensor 150. As an example, first piezoelectric sensor or transducer 154 and second piezoelectric sensor or transducer 156 may each be configured to operate as both an ultrasonic transmitter and an ultrasonic receiver. In such an example, ultrasonic flow sensor 150 may be configured to operate by alternately transmitting and receiving a burst of ultrasound between the two transducers (e.g., “up” and “down” signals, etc.) by measuring the transit time that it takes for sound to travel between the two transducers in both directions. An analog-to-digital converter (ADC) may sample the signal received at the receiving transducer at a plurality of time points to generate a time-series that includes the plurality of amplitudes sampled at the plurality of time points. For example, FIG. 6 is a graph of example “up” and “down” time series signals received at the receiving transducers showing offset due to fluid flow. The difference in the transit time (e.g., A time, etc.) measured may be directly proportional to a velocity of the fluid in the fluid flow path. A plurality of differences in transit time (e.g., a plurality of A times, etc.) may be represented as a time-series that includes at least one of a plurality of amplitudes at a plurality of time points, a plurality of phases at the plurality of time points, or any combination thereof. For example, an average Δt may be calculated over one or more “up” and “down” signal pairs or cycles, the magnitude of which may be proportional to the flow rate of the fluid through the fluid flow path of ultrasonic flow sensor 150. As an example, a Δt may be converted to a velocity of the fluid in flow tube 102 using an angle of the ultrasound signal path, which may be calculated by knowing the speed of sound of flow tube 102 and the fluid. This angle may be used with trigonometry to convert the ultrasound path into a straight line in flow tube 102, which may be the velocity of the liquid in flow tube 102. The velocity of the fluid may be converted into a flow rate by multiplying the velocity by a cross-sectional area of the pipe, and a volume of the fluid delivered may be calculated by multiplying the flow rate by time.

Ultrasonic flow sensor 150 may be configured to continually generate and provide the time-series corresponding to the flow of at least one fluid through a fluid flow path of ultrasonic flow sensor 150 during the flow of at least one fluid through a fluid flow path of ultrasonic flow sensor 150 (e.g., as the flow of at least one fluid through a fluid flow path of ultrasonic flow sensor 150 occurs and progresses, etc.).

Flow sensor system 102 and/or external computing system 104 may be configured to alternately provide an excitation pulse pattern including a number of excitation pulses to first piezoelectric sensor or transducer 154 and second piezoelectric sensor or transducer 156 to cause first piezoelectric sensor or transducer 154 and second piezoelectric sensor or transducer 156 to alternately transmit and receive the burst of ultrasound (e.g., a ultrasonic signal, etc.) between the two transducers. For example, first piezoelectric sensor or transducer 154 may be configured to transmit a first ultrasonic signal to second piezoelectric sensor or transducer 156, second piezoelectric sensor or transducer 156 may be configured to transmit a second ultrasonic signal to first piezoelectric transducer 156, second piezoelectric sensor or transducer 156 may be configured to receive the first ultrasonic signal transmitted by first piezoelectric sensor or transducer 154 (which may be sampled by an ADC to generate the time-series), and/or first piezoelectric sensor or transducer 156 may be configured to receive the second ultrasonic signal transmitted by second piezoelectric sensor or transducer 156 (which may be sampled by an ADC to generate the time-series).

External computing system 104 may include one or more devices capable of receiving information and/or data from flow sensor system 102 and/or communicating information and/or data to flow sensor system 102. For example, external computing system 104 may include one or more computing systems including one or more processors (e.g., one or more computing devices, one or more mobile computing devices, one or more servers, etc.). In some non-limiting embodiments or aspects, external computing system 104 includes a nurse station in a hospital, a hospital information system (HIS), an electronic medical records (EMR) system, a radiology information system (RIS), a picture archiving and communication system (PACS), a laboratory information system (LIS), a smart phone, a tablet computer, any combination thereof, and/or the like.

The number and arrangement of systems and devices shown in FIGS. 1A-1C are provided as an example. There may be additional systems or devices, fewer systems or devices, different systems or devices, or differently arranged systems or devices than those shown in FIGS. 1A-1C. Furthermore, two or more systems or devices shown in FIGS. 1A-1C may be implemented within a single system or device, or a single system or device shown in FIGS. 1A-1C may be implemented as multiple, distributed systems or devices. Additionally or alternatively, a set of systems (e.g., one or more systems) or a set of devices (e.g., one or more devices) of system 100 may perform one or more functions described as being performed by another set of systems or another set of devices of system 100.

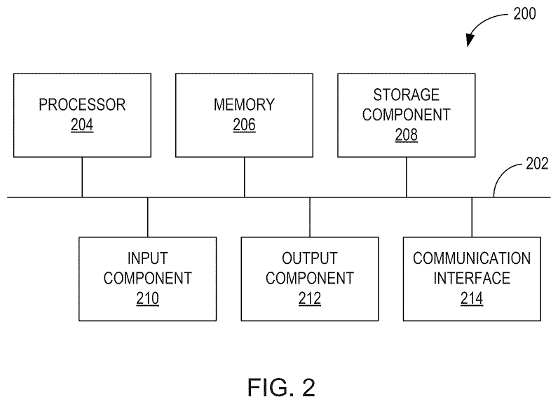

Referring now to FIG. 2, shown is a diagram of example components of a device 200 according to non-limiting embodiments. Device 200 may correspond to flow sensor system 102 and/or external computing system 104 in FIG. 1A, as an example. In some non-limiting embodiments, such systems or devices may include at least one device 200 and/or at least one component of device 200. The number and arrangement of components shown are provided as an example. In some non-limiting embodiments, device 200 may include additional components, fewer components, different components, or differently arranged components than those shown. Additionally, or alternatively, a set of components (e.g., one or more components) of device 200 may perform one or more functions described as being performed by another set of components of device 200.

As shown in FIG. 2, device 200 may include a bus 202, a processor 204, memory 206, a storage component 208, an input component 210, an output component 212, and a communication interface 214. Bus 202 may include a component that permits communication among the components of device 200. In some non-limiting embodiments, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), or any processing component (e.g., a field-programmable gate array (FPGA), an application-specific integrated circuit (ASIC), etc.) that can be programmed to perform a function. Memory 206 may include random access memory (RAM), read only memory (ROM), or another type of dynamic or static storage device (e.g., flash memory, magnetic memory, optical memory, etc.) that stores information and/or instructions for use by processor 204.

With continued reference to FIG. 2, storage component 208 may store information and/or software related to the operation and use of device 200. For example, storage component 208 may include a hard disk (e.g., a magnetic disk, an optical disk, a magneto-optic disk, a solid-state disk, etc.) or another type of computer-readable medium. Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.). Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, or the like.

Device 200 may perform one or more processes described herein. Device 200 may perform these processes based on processor 204 executing software instructions stored by a computer-readable medium, such as memory 206 or storage component 208. A computer-readable medium may include any non-transitory memory device. A memory device includes memory space located inside of a single physical storage device or memory space spread across multiple physical storage devices. Software instructions may be read into memory 206 and/or storage component 208 from another computer-readable medium or from another device via communication interface 214. When executed, software instructions stored in memory 206 or storage component 208 may cause processor 204 to perform one or more processes described herein. Additionally, or alternatively, hardwired circuitry may be used in place of or in combination with software instructions to perform one or more processes described herein. Thus, embodiments described herein are not limited to any specific combination of hardware circuitry and software. The term “configured to,” as used herein, may refer to a specific arrangement of software, device(s), or hardware for performing or enabling one or more of the innovative functions (e.g., actions, processes, steps of a process, or the like) described herein. For example, “a processor configured to” may refer to a processor that executes specific software instructions (e.g., program code) that cause the processor to perform one or more functions related to fluid flow detection and/or identification.

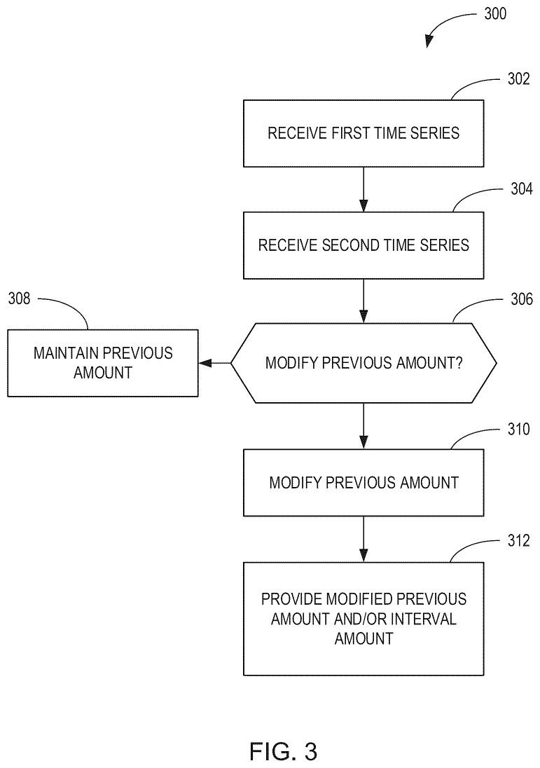

Referring now to FIG. 3, shown is a flow diagram for a method 300 for inhibiting spurious flow measurement by ultrasonic flow sensors, according to some non-limiting embodiments or aspects. The steps shown in FIG. 3 are for example purposes only. It will be appreciated that additional, fewer, different, or a different order of steps may be used in some non-limiting embodiments or aspects. In some non-limiting embodiments or aspects, a step may be automatically performed in response to performance or completion of a prior step.

As shown in FIG. 3, at step 302, method 300 includes receiving a first time-series generated by the ultrasonic flow sensor by causing the first piezoelectric sensor or transducer to transmit at least one first ultrasonic signal to the second piezoelectric sensor or transducer. For example, flow sensor system 102 and/or external computing system 104 may receive a first time-series generated by ultrasonic flow sensor 150 by causing first piezoelectric sensor or transducer 154 to transmit at least one first ultrasonic signal to second piezoelectric sensor or transducer 156. The first time series may include a plurality of first amplitudes of the at least one first ultrasonic signal received at second piezoelectric sensor or transducer 156 at a plurality of first time points in a time interval.

As shown in FIG. 3, at step 304, method 300 includes receiving a second time-series generated by the ultrasonic flow sensor by causing the second piezoelectric sensor or transducer to transmit at least one second ultrasonic signal to the first piezoelectric sensor or transducer. For example, flow sensor system 102 and/or external computing system 104 may receive a second time-series generated by ultrasonic flow sensor 150 by causing second piezoelectric sensor or transducer 156 to transmit at least one second ultrasonic signal to first piezoelectric sensor or transducer 154. The second time series may include a plurality of second amplitudes of the at least one second ultrasonic signal received at first piezoelectric sensor or transducer 154 at a plurality of second time points in the time interval. The first time series and the second time series may be in the same time interval.

As shown in FIG. 3, at step 306, method 300 includes determining, based on the first time-series and the second time-series, whether to modify a previous amount associated with at least one attribute of the fluid with an interval amount associated with the at least one attribute of the fluid. For example, flow sensor system 102 and/or external computing system 104 may determine, based on the first time-series and the second time-series, whether to modify a previous amount associated with at least one attribute of the fluid with an interval amount associated with the at least one attribute of the fluid. As an example, flow sensor system 102 and/or external computing system 104 may determine whether to modify the previous amount associated with the at least one attribute of the fluid with the interval amount associated with the at least one attribute of the fluid based (e.g., determine whether the interval amount is associated with a spurious flow, etc.) on at least one of the following: (i) the plurality of first amplitudes and the plurality of second amplitudes, (ii) the plurality of first time points and the plurality of second time points, or any combination thereof.

The at least one attribute of the fluid may include at least one of the following attributes of the fluid: a flow rate of the fluid in a fluid flow path of ultrasonic flow sensor 150 defined by flow tube 152, a volume of the fluid delivered (e.g., to a patient, etc.) though the fluid flow path of ultrasonic flow sensor 150, or any combination thereof. Flow sensor system 102 and/or external computing system 104 may calculate the flow rate of the fluid in the fluid flow path of ultrasonic flow sensor 150 defined by flow tube 152 and/or the volume of the fluid delivered though the fluid flow path of ultrasonic flow sensor 150 as previously described herein with respect to FIGS. 1C and 6.

The previous amount associated with the at least one attribute of the fluid may include a volume (e.g., an accumulated volume, etc.) of the fluid delivered through the fluid flow path of ultrasonic flow sensor in one or more previous time intervals and/or a flow rate (e.g., an average flow rate, etc.) of the fluid in a fluid flow path of ultrasonic flow sensor 150 defined by flow tube 152 in the one or more previous time intervals. The interval amount associated with the at least one attribute of the fluid may include volume of the fluid delivered through the fluid flow path of ultrasonic flow sensor in the time interval and/or a flow rate (e.g., an average flow rate, etc.) of the fluid in a fluid flow path of ultrasonic flow sensor 150 defined by flow tube 152 in the time interval. The interval amount associated with the at least one attribute of the fluid may be calculated based on the first time-series and the second-time series.

In some non-limiting embodiments or aspects, flow sensor system 102 and/or external computing system 104 may determine, based on the first time-series and the second time-series, whether to modify the previous amount associated with the at least one attribute of the fluid with the interval amount associated with the at least one attribute of the fluid by: providing, as input to at least one machine learning model, the first time-series and the second time-series; and receiving, as output from the at least one machine learning model, a prediction of whether the flow is spurious (e.g., an indication of whether to modify the previous amount associated with the at least one attribute of the fluid with the interval amount associated with the at least one attribute, etc.). The at least one machine learning model (e.g., a neural network, a recurrent neural network, a convolutional neural network, etc.) may be trained using machine learning or other artificial intelligence techniques to accept, as input, the first time series and the second time series (e.g., a feature representation associated with the first time series and the second time series, etc.) and provide, as output, the prediction of whether the flow is spurious.

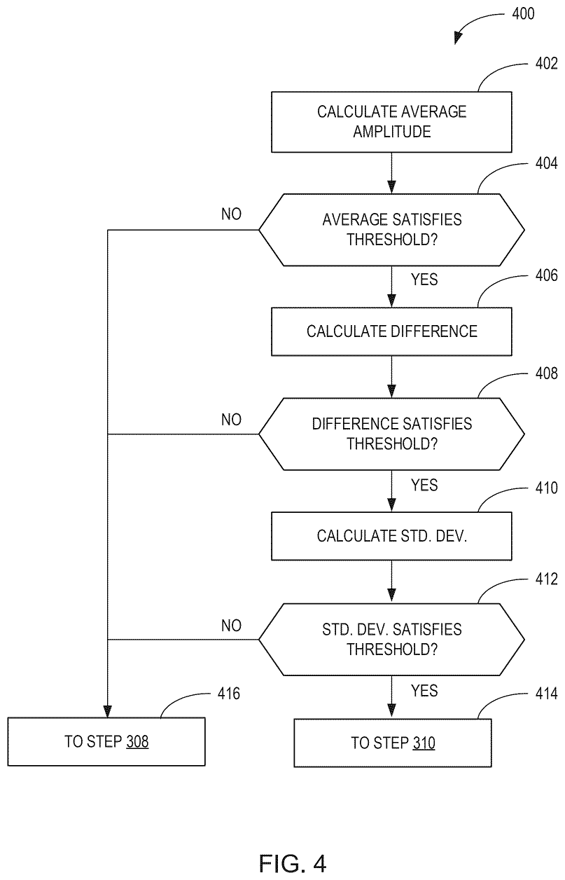

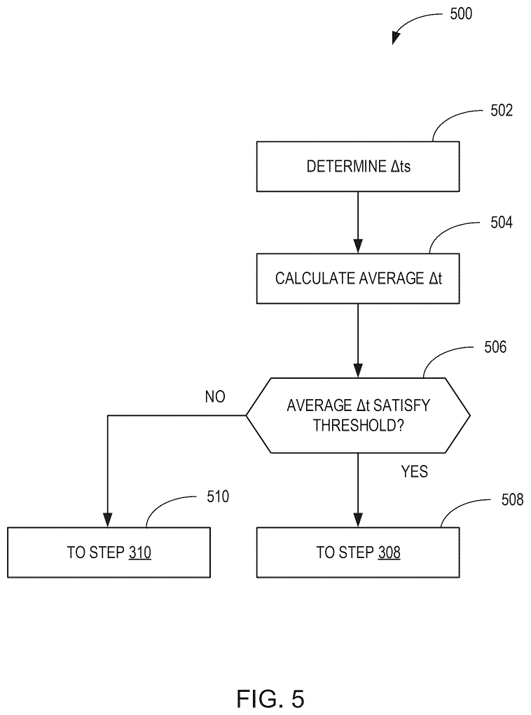

Further details regarding non-limiting embodiments or aspects of step 306 of method 300 are provided below with regard to FIGS. 4 and 5.

As shown in FIG. 3, at step 308, method 300 includes, in response to determining not to modify the previous amount associated with the at least one attribute of the fluid with the interval amount associated with the at least one attribute of the fluid, maintaining the previous amount associated with the at least one attribute of the fluid. For example, flow sensor system 102 and/or external computing system 104 may, in response to determining not to modify the previous amount associated with the at least one attribute of the fluid with the interval amount associated with the at least one attribute of the fluid, maintain the previous amount associated with the at least one attribute of the fluid. As an example, flow sensor system 102 and/or external computing system 104 may flag the interval amount associated with the at least one attribute of the fluid as spurious flow without updating a continual calculation of the volume delivered, the average flow rate, or the like.

In some non-limiting embodiments or aspects, flow sensor system 102 and/or external computing system 104 may maintain the previous amount associated with the at least one attribute of the fluid and provide an indication associated with the determination that the interval amount is associated with a spurious flow (e.g., an indication that spurious flow is detected, etc.). The indication may be a human perceivable output indicating the modified previous amount and/or the interval amount (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval (e.g., in a memory of ultrasonic flow sensor 150, in a memory of flow sensory system, in a database etc.), transmitting a value directly to the recipient via at least one wired or wireless communication medium, transmitting or storing a reference to a value, and the like. The providing of the indication may additionally or alternatively include encoding, decoding, encrypting, decrypting, validating, verifying, and the like via a hardware element.

As shown in FIG. 3, at step 310, method 300 includes, in response to determining to modify the previous amount associated with the at least one attribute of the fluid with the interval amount associated with the at least one attribute of the fluid, modifying the previous amount associated with the at least one attribute of the fluid with the interval amount associated with the at least one attribute of the fluid. For example, flow sensor system 102 and/or external computing system 104 may, in response to determining to modify the previous amount associated with the at least one attribute of the fluid with the interval amount associated with the at least one attribute of the fluid, modify the previous amount associated with the at least one attribute of the fluid with the interval amount associated with the at least one attribute of the fluid. As an example, flow sensor system 102 and/or external computing system 104 may, in response to determining to modify the previous amount associated with the at least one attribute of the fluid with the interval amount associated with the at least one attribute of the fluid, add the interval amount associated with the at least one attribute of the fluid to the previous amount associated with the at least one attribute of the fluid (e.g., increase a previous volume by an interval volume, add an interval flow rate to a calculation of an average flow rate including the previous flow rate, etc.).

As shown in FIG. 3, at step 312, method 300 includes providing an indication associated with the modified previous amount associated with the at least one attribute of the fluid and/or the interval amount associated with the at least one attribute of the fluid. For example, glow sensor system 102 and/or external computing system 104 may provide an indication associated with the modified previous amount associated with the at least one attribute of the fluid (e.g., an updated volume delivered, an updated average flow rate, etc.) and/or the interval amount associated with the at least one attribute of the fluid. The indication may be a human perceivable output indicating the modified previous amount and/or the interval amount (e.g., provided via a speaker and/or a display of a reusable base of the flow sensor system in U.S. Patent Application Publication No. 2021/0231471, etc.). In some implementations, providing the indication may include storing a value in a location of a storage device for subsequent retrieval (e.g., in a memory of ultrasonic flow sensor 150, in a memory of flow sensory system, in a database etc.), transmitting a value directly to the recipient via at least one wired or wireless communication medium, transmitting or storing a reference to a value, and the like. The providing at step 312 may additionally or alternatively include encoding, decoding, encrypting, decrypting, validating, verifying, and the like via a hardware element.

In some non-limiting embodiments or aspects, flow sensor system 102 and/or external computing system 104 may provide the indication associated with the modified previous amount associated with the at least one attribute of the fluid (e.g., the updated volume delivered, the updated average flow rate, etc.) and/or the interval amount associated with the at least one attribute of the fluid in association with patient data associated with a patient, procedure data associated with a patient procedure associated with the patient, caregiver data associated with a caregiver (e.g., a nurse, a doctor, etc.), any combination thereof, or the like. Patient data associated with a patient may include a patient identifier associated with the patient (e.g., a unique patient identifier, etc.), patient demographics (e.g., a name, an age, a sex, a weight, a height, a birthdate, an address, etc.), a list of medication allergies associated with the patient, a list of medication doses delivered, being delivered, and/or pending for delivery to the patient, any combination thereof, or the like. Procedure data may include a procedure identifier associated with the procedure (e.g., a unique procedure identifier, etc.), one or more medical devices associated with the procedure, a name of the procedure, a state of the procedure (e.g., scheduled for a future date and time, currently being performed, previously performed a previous date and time, etc.), a caregiver associated with the procedure, a patient associated with the procedure, any combination thereof, or the like. Caregiver data may include a caregiver identifier associated with the caregiver (e.g., a unique caregiver identifier, etc.), a name of the caregiver, any combination thereof, or the like.

Referring now to FIG. 4, shown is a flow diagram for a method 400 for inhibiting spurious flow measurement by ultrasonic flow sensors, according to some non-limiting embodiments or aspects. The steps shown in FIG. 4 are for example purposes only. It will be appreciated that additional, fewer, different, or a different order of steps may be used in some non-limiting embodiments or aspects. In some non-limiting embodiments or aspects, a step may be automatically performed in response to performance or completion of a prior step.

As shown in FIG. 4, at step 402, method 400 includes calculating, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval. For example, flow sensor system 102 and/or external computing system 104 may calculate, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval. As an example, flow sensor system 102 and/or external computing system 104 may calculate a first average amplitude of the plurality of first amplitudes of the at least one first ultrasonic signal received at second piezoelectric sensor or transducer 156 at the plurality of first time points in the time interval, a second average amplitude of the plurality of second amplitudes of the at least one second ultrasonic signal received at first piezoelectric sensor or transducer 154 at the plurality of second time points in the time interval, and an average amplitude of the first average amplitude and the second average amplitude.

As shown in FIG. 4, at step 404, method 400 includes determining whether the average amplitude satisfies a threshold amplitude. For example, flow sensor system 102 and/or external computing system 104 may determine whether the average amplitude satisfies a threshold amplitude. The threshold may be a static threshold stored by or accessible to the system or device executing step 404. The threshold may be a dynamic threshold that is generated based on information stored by or accessible to the system or device executing step 404. For example, the threshold may be based on the fluid being administered (e.g., a look up table of thresholds indexed by fluid or fluid types). Alternatively, the fluid may have certain properties such as refractive index, viscosity, or the like that may be provided to a threshold generator to calculate a threshold based on the input values.

As shown in FIG. 4, at step 406, method 400 includes, in response to determining that the average amplitude satisfies the threshold amplitude at step 404 of method 400, calculating, based on the first time series and the second time series, a relative difference between the first time series and the second time series. For example, flow sensor system 102 and/or external computing system 104 may, in response to determining that the average amplitude satisfies the threshold amplitude at step 404 of method 400, calculate, based on the first time series and the second time series, a relative difference between the first time series and the second time series. As an example, flow sensor system 102 and/or external computing system 104 may, in response to determining that the average amplitude satisfies the threshold amplitude at step 404 of method 400, calculate the relative difference between the first time series and the second time series as the absolute value of the first average amplitude minus the second average amplitude multiplied by a constant (e.g., 100, etc.) and divided by the average amplitude of the first average amplitude and the second average amplitude. In such an example, the relative difference between the first time series and the second time series may include a percentage difference.

As shown in FIG. 4, at step 408, method 400 includes determining whether the relative difference satisfies a threshold difference. For example, flow sensor system 102 and/or external computing system 104 may determine whether the relative difference satisfies a threshold difference (e.g., 6%, etc.). The threshold difference may be a static threshold difference stored by or accessible to the system or device executing step 408. The threshold difference may be a dynamic threshold difference that is generated based on information stored by or accessible to the system or device executing step 408. For example, the threshold difference may be based on the fluid being administered (e.g., a look up table of threshold differences indexed by fluid or fluid types). Alternative, the fluid may have certain properties such as refractive index, viscosity, or the like that may be provided to a threshold difference generator to calculate a threshold difference based on the input values. In some implementations, the threshold difference may be set based on a user configuration. For example, for certain fluids or patients, the threshold difference may be configured narrowly to detect small differences while for other patients or fluids, the threshold difference may be a wider range and still be clinically appropriate. The configuration may be performed via a user interface to receive the specific threshold difference or selection from a list of sensitivity levels, each level corresponding to a different threshold difference.

As shown in FIG. 4, at step 410, method 400 includes, in response to determining that the relative difference satisfies the threshold difference at step 408 of method 400, calculating, based on the first time series and the second time series, a standard deviation of a flow rate of the fluid in the flow tube over a plurality of time intervals before the time interval. For example, flow sensor system 102 and/or external computing system 104 may, in response to determining that the relative difference satisfies the threshold difference at step 408 of method 400, calculate, based on the first time series and the second time series, a standard deviation of a flow rate of the fluid in flow tube 152 over a plurality of time intervals before the time interval.

As shown in FIG. 4, at step 412, method 400 includes determining whether the standard deviation of the flow rate of the fluid in the flow tube over the plurality of time intervals before the time interval satisfies a threshold deviation. For example, flow sensor system 102 and/or external computing system 104 may determine whether the standard deviation of the flow rate of the fluid in the flow tube over the plurality of time intervals before the time interval satisfies a threshold deviation. The threshold difference may be a static threshold difference stored by or accessible to the system or device executing step 412. The threshold difference may be a dynamic threshold difference that is generated based on information stored by or accessible to the system or device executing step 412. For example, the threshold difference may be based on the fluid being administered (e.g., a look up table of threshold differences indexed by fluid or fluid types). Alternative, the fluid may have certain properties such as refractive index, viscosity, or the like that may be provided to a threshold difference generator to calculate a threshold difference based on the input values. In some implementations, the threshold difference may be set based on a user configuration. For example, for certain fluids or patients, the threshold difference may be configured narrowly to detect small differences while for other patients or fluids, the threshold difference may be a wider range and still be clinically appropriate. The configuration may be performed via a user interface to receive the specific threshold difference or selection from a list of sensitivity levels, each level corresponding to a different threshold difference.

As shown in FIG. 4, at step 414, method 400 includes, (i) in response to determining that the standard deviation of the flow rate of the fluid in the flow tube satisfies the threshold deviation at step 412 of method 400, (ii) in response to determining that the average amplitude satisfies the threshold amplitude at step 404 of method 400, (iii) in response to determining that the relative difference satisfies the threshold difference at step 408 of method 400, or any combination thereof, proceeding processing to step 310 of method 300 to modify the previous amount associated with the at least one attribute of the fluid with the interval amount associated with the at least one attribute of the fluid. For example, flow sensor system 102 and/or external computing system 104 may, (i) in response to determining that the standard deviation of the flow rate of the fluid in the flow tube satisfies the threshold deviation at step 412 of method 400, (ii) in response to determining that the average amplitude satisfies the threshold amplitude at step 404 of method 400, (iii) in response to determining that the relative difference satisfies the threshold difference at step 408 of method 400, or any combination thereof, determine at step 306 of method 300 to modify the previous amount associated with the at least one attribute of the fluid with the interval amount associated with the at least one attribute of the fluid (e.g., determine that the flow associate with the interval amount is not spurious flow, etc.).

As shown in FIG. 4, at step 416, method 400 includes, in response to determining that the average amplitude does not satisfy the threshold amplitude at step 404 of method 400, in response to determining that the relative difference does not the threshold difference at step 408 of method 400, or in response to determining that the standard deviation of the flow rate of the fluid in the flow tube satisfies the threshold deviation at step 412 of method 400, proceeding processing to step 308 of method 300 to maintain the previous amount associated with the at least one attribute of the fluid. For example, flow sensor system 102 and/or external computing system 104 may, in response to determining that the average amplitude does not satisfy the threshold amplitude at step 404 of method 400, in response to determining that the relative difference does not the threshold difference at step 408 of method 400, or in response to determining that the standard deviation of the flow rate of the fluid in the flow tube satisfies the threshold deviation at step 412 of method 400, determine at step 306 of method 300 to maintain the previous amount associated with the at least one attribute of the fluid (e.g., determine that the flow associate with the interval amount is spurious flow, etc.).

Referring now to FIG. 5, shown is a flow diagram for a method 500 for inhibiting spurious flow measurement by ultrasonic flow sensors, according to some non-limiting embodiments or aspects. The steps shown in FIG. 5 are for example purposes only. It will be appreciated that additional, fewer, different, or a different order of steps may be used in some non-limiting embodiments or aspects. In some non-limiting embodiments or aspects, a step may be automatically performed in response to performance or completion of a prior step.

As shown in FIG. 5, at step 502, method 500 includes determining, based on the first time series and the second time series, a plurality of differences in transit time Δt between the at least one first ultrasonic signal and the at least one second ultrasonic signal. For example, flow sensor system 102 and/or external computing system 104 may determine, based on the first time series and the second time series, a plurality of differences in transit time Δt between the at least one first ultrasonic signal and the at least one second ultrasonic signal. As an example, flow sensor system 102 and/or external computing system 104 may determine, based on the first time series and the second time series, the plurality of differences in transit time Δt between the at least one first ultrasonic signal and the at least one second ultrasonic signal as previously described herein with respect to FIGS. 1C and 6.

As shown in FIG. 5, at step 504, method 500 includes calculating an average difference in transit time Δt of the plurality of differences in transit time Δt. For example, flow sensor system 102 and/or external computing system 104 may calculate an average difference in transit time Δt of the plurality of differences in transit time Δt. As an example, the average difference in in transit time Δt of the plurality of differences in transit time Δt may be calculated as a summation of the plurality of differences in transit time Δt divided by a number of the plurality of differences in transit time Δt.

As shown in FIG. 5, at step 506, method 500 includes determining whether a range of the differences in transit time Δt between the at least one first ultrasonic signal and the at least one second ultrasonic signal satisfies a threshold range determined based on the average difference in transit time Δt of the plurality of differences in transit time Δt. For example, flow sensor system 102 and/or external computing system 104 may determine whether a range of the differences in transit time Δt between the at least one first ultrasonic signal and the at least one second ultrasonic signal satisfies a threshold range determined based on the average difference in transit time Δt of the plurality of differences in transit time Δt. A range of the differences in transit time Δt between the at least one first ultrasonic signal and the at least one second ultrasonic signal may be defined by a maximum difference in transit time Δt of the plurality of differences in transit time and a minimum difference in transit time Δt of the plurality of differences in transit time. The threshold range may be determined based on the average difference in transit time Δt of the plurality of differences in transit time Δt multiplied by a constant (e.g., 0.5, etc.). As an example, in a case of real flow, a spread in different Δts may be smaller, but in a case of spurious flow, the spread may be a significant percentage of average Δt.

As shown in FIG. 5, at step 508, method 500 includes, in response to determining that the range of the differences in transit time Δt between the at least one first ultrasonic signal and the at least one second ultrasonic signal satisfies the threshold range determined based on the average difference in transit time Δt of the plurality of differences in transit time Δt, proceeding processing to step 310 of method 300 to modify the previous amount associated with the at least one attribute of the fluid with the interval amount associated with the at least one attribute of the fluid. For example, flow sensor system 102 and/or external computing system 104 may, in response to determining that the range of the differences in transit time Δt between the at least one first ultrasonic signal and the at least one second ultrasonic signal satisfies the threshold range determined based on the average difference in transit time Δt of the plurality of differences in transit time Δt, determine at step 306 of method 300 to modify the previous amount associated with the at least one attribute of the fluid with the interval amount associated with the at least one attribute of the fluid (e.g., determine that the flow associate with the interval amount is not spurious flow, etc.).

As shown in FIG. 5, at step 510, method 500 includes, in response to determining that the range of the differences in transit time Δt between the at least one first ultrasonic signal and the at least one second ultrasonic signal does not satisfy the threshold range determined based on the average difference in transit time Δt of the plurality of differences in transit time Δt, proceeding processing to step 308 of method 300 to maintain the previous amount associated with the at least one attribute of the fluid. For example, flow sensor system 102 and/or external computing system 104 may, in response to determining that the range of the differences in transit time Δt between the at least one first ultrasonic signal and the at least one second ultrasonic signal does not satisfy the threshold range determined based on the average difference in transit time Δt of the plurality of differences in transit time Δt, determine at step 306 of method 300 to maintain the previous amount associated with the at least one attribute of the fluid (e.g., determine that the flow associate with the interval amount is spurious flow, etc.).

Although method 500 is described herein with respect to FIG. 5 and method 400 is described herein with respect to FIG. 4, it will be appreciated that method 500 may be used in combination with method 400 in some non-limiting embodiments or aspects.

Accordingly, non-limiting embodiments or aspects of the present disclosure may use characteristics of ultrasonic flow sensor signals as a screen or filter in a flow-algorithm to determine whether flow is real or spurious. In case of spurious flow, non-limiting embodiments or aspects of the present disclosure may ignore a corresponding calculated flow and a value associated therewith displayed on a display unit may remain unchanged.

Although embodiments have been described in detail for the purpose of illustration, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

Aspects described include artificial intelligence or other operations whereby the system processes inputs and generates outputs with apparent intelligence. The artificial intelligence may be implemented in whole or in part by a model. A model may be implemented as a machine learning model. The learning may be supervised, unsupervised, reinforced, or a hybrid learning whereby multiple learning techniques are employed to generate the model. The learning may be performed as part of training. Training the model may include obtaining a set of training data and adjusting characteristics of the model to obtain a desired model output. For example, three characteristics may be associated with a desired item location. In such instance, the training may include receiving the three characteristics as inputs to the model and adjusting the characteristics of the model such that for each set of three characteristics, the output device state matches the desired device state associated with the historical data.

In some implementations, the training may be dynamic. For example, the system may update the model using a set of events. The detectable properties from the events may be used to adjust the model.

The model may be an equation, artificial neural network, recurrent neural network, convolutional neural network, decision tree, or other machine-readable artificial intelligence structure. The characteristics of the structure available for adjusting during training may vary based on the model selected. For example, if a neural network is the selected model, characteristics may include input elements, network layers, node density, node activation thresholds, weights between nodes, input or output value weights, or the like. If the model is implemented as an equation (e.g., regression), the characteristics may include weights for the input parameters, thresholds, or limits for evaluating an output value, or criterion for selecting from a set of equations.

Once a model is trained, retraining may be included to refine or update the model to reflect additional data or specific operational conditions. The retraining may be based on one or more signals detected by a device described herein or as part of a method described herein. Upon detection of the designated signals, the system may activate a training process to adjust the model as described.

Further examples of machine learning and modeling features which may be included in the embodiments discussed above are described in “A survey of machine learning for big data processing” by Qiu et al. in EURASIP Journal on Advances in Signal Processing (2016) which is hereby incorporated by reference in its entirety.

What is claimed is:

1. A system comprising:
an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube; and
at least one processor configured to:
receive a first time-series generated by the ultrasonic flow sensor by causing the first piezoelectric sensor or transducer to transmit at least one first ultrasonic signal to the second piezoelectric sensor or transducer;
receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and
determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

2. The system of claim 1, wherein the at least one attribute of the fluid includes at least one of the following attributes of the fluid: a flow rate, a volume, or any combination thereof.

3. The system of claim 1, wherein the first time series includes a plurality of first amplitudes of the at least one first ultrasonic signal received at the second piezoelectric sensor or transducer at a plurality of first time points in the time interval,
wherein the second time series includes a plurality of second amplitudes of the at least one second ultrasonic signal received at the first piezoelectric sensor or transducer at a plurality of second time points in the time interval, and
wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

4. The system of claim 3, wherein the at least one processor is configured to determine, based on the first time-series and the second time-series, whether to modify the previous amount associated with at least one attribute of the fluid with the interval amount associated with the at least one attribute of the fluid by:
calculating, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and
determining whether the average amplitude satisfies a threshold amplitude.

5. The system of claim 3, wherein the at least one processor is configured to determine, based on the first time-series and the second time-series, whether to modify the previous amount associated with at least one attribute of the fluid with the interval amount associated with the at least one attribute of the fluid by:
calculating, based on the first time series and the second time series, a relative difference between the first time series and the second time series; and
determining whether the relative difference between the first time series and the second time series satisfies a threshold difference.

6. The system of claim 3, wherein the at least one processor is configured to determine, based on the first time-series and the second time-series, whether to modify the previous amount associated with at least one attribute of the fluid with the interval amount associated with the at least one attribute of the fluid by:
calculating a standard deviation of a flow rate of the fluid in the flow tube over a plurality of time intervals before the time interval; and
determining whether the standard deviation of the flow rate of the fluid in the flow tube satisfies a threshold deviation.

7. The system of claim 3, wherein the at least one processor is configured to determine, based on the first time-series and the second time-series, whether to modify the previous amount associated with at least one attribute of the fluid with the interval amount associated with the at least one attribute of the fluid by:
determining, based on the first time series and the second time series, a plurality of differences in transit time between the at least one first ultrasonic signal and the at least one second ultrasonic signal;
calculating an average difference in transit time of the plurality of differences in transit time; and
determining whether a range of the differences in transit time between the at least one first ultrasonic signal and the at least one second ultrasonic signal satisfies a threshold range determined based on the average difference in transit time of the plurality of differences in transit time.

8. The system of claim 3, wherein the at least one processor is configured to determine, based on the first time-series and the second time-series, whether to modify the previous amount associated with at least one attribute of the fluid with the interval amount associated with the at least one attribute of the fluid by:
providing, as input to at least one machine learning model, the first time-series and the second time-series; and
receiving, as output from the at least one machine learning model, a prediction of whether to modify the previous amount associated with the at least one attribute of the fluid with the interval amount associated with the at least one attribute.

9. An ultrasonic flow sensor comprising:
a flow tube for delivering a fluid from a fluid source;
a first piezoelectric sensor or transducer arranged at an upstream position of the flow tube;
a second piezoelectric sensor or transducer arranged at a downstream position of the flow tube; and
at least one processor configured to:
receive a first time-series generated by the ultrasonic flow sensor by causing the first piezoelectric sensor or transducer to transmit at least one first ultrasonic signal to the second piezoelectric sensor or transducer;
receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and
determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

10. The ultrasonic flow sensor of claim 9, wherein the first time series includes a plurality of first amplitudes of the at least one first ultrasonic signal received at the second piezoelectric sensor or transducer at a plurality of first time points in the time interval,
wherein the second time series includes a plurality of second amplitudes of the at least one second ultrasonic signal received at the first piezoelectric sensor or transducer at a plurality of second time points in the time interval, and
wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

11. A method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube, the method comprising:
receiving, with at least one processor, a first time-series generated by the ultrasonic flow sensor by causing the first piezoelectric sensor or transducer to transmit at least one first ultrasonic signal to the second piezoelectric sensor or transducer;
receiving, with the at least one processor, a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and
determining, with the at least one processor, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

12. The method of claim 11, wherein the at least one attribute of the fluid includes at least one of the following attributes of the fluid: a flow rate, a volume, or any combination thereof.

13. The method of claim 11, wherein the first time series includes a plurality of first amplitudes of the at least one first ultrasonic signal received at the second piezoelectric sensor or transducer at a plurality of first time points in the time interval,
wherein the second time series includes a plurality of second amplitudes of the at least one second ultrasonic signal received at the first piezoelectric sensor or transducer at a plurality of second time points in the time interval, and
wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.

14. The method of claim 13, wherein determining, with the at least one processor, based on the first time-series and the second time-series, whether to modify the previous amount associated with at least one attribute of the fluid with the interval amount associated with the at least one attribute of the fluid includes:
calculating, with the at least one processor, based on the first time series and the second time series, an average amplitude associated with the at least one first ultrasonic signal received at the second piezoelectric sensor or transducer at the plurality of first time points in the time interval and the at least one second ultrasonic signal received at the first piezoelectric sensor or transducer at the plurality of second time points in the time interval; and
determining, with the at least one processor, whether the average amplitude satisfies a threshold amplitude.

15. The method of claim 13, wherein determining, with the at least one processor, based on the first time-series and the second time-series, whether to modify the previous amount associated with at least one attribute of the fluid with the interval amount associated with the at least one attribute of the fluid includes:
calculating, with the at least one processor, based on the first time series and the second time series, a relative difference between the first time series and the second time series; and
determining, with the at least one processor, whether the relative difference between the first time series and the second time series satisfies a threshold difference.

16. The method of claim 13, wherein determining, with the at least one processor, based on the first time-series and the second time-series, whether to modify the previous amount associated with at least one attribute of the fluid with the interval amount associated with the at least one attribute of the fluid includes:
calculating, with the at least one processor, a standard deviation of a flow rate of the fluid in the flow tube over a plurality of time intervals before the time interval; and
determining, with the at least one processor, whether the standard deviation of the flow rate of the fluid in the flow tube satisfies a threshold deviation.

17. The method of claim 13, wherein determining, with the at least one processor, based on the first time-series and the second time-series, whether to modify the previous amount associated with at least one attribute of the fluid with the interval amount associated with the at least one attribute of the fluid includes:
determining, with the at least one processor, based on the first time series and the second time series, a plurality of differences in transit time between the at least one first ultrasonic signal and the at least one second ultrasonic signal;
calculating, with the at least one processor, an average difference in transit time of the plurality of differences in transit time; and
determining, with the at least one processor, whether a range of the differences in transit time between the at least one first ultrasonic signal and the at least one second ultrasonic signal satisfies a threshold range determined based on the average difference in transit time of the plurality of differences in transit time.

18. The system of claim 13, wherein determining, with the at least one processor, based on the first time-series and the second time-series, whether to modify the previous amount associated with at least one attribute of the fluid with the interval amount associated with the at least one attribute of the fluid includes:
providing, with the at least one processor, as input to at least one machine learning model, the first time-series and the second time-series; and
receiving, with the at least one processor, as output from the at least one machine learning model, a prediction of whether to modify the previous amount associated with the at least one attribute of the fluid with the interval amount associated with the at least one attribute.

19. A computer program product including a non-transitory computer readable medium including program instructions method for inhibiting spurious flow measurement by an ultrasonic flow sensor that includes a flow tube for delivering a fluid from a fluid source, a first piezoelectric sensor or transducer arranged at an upstream position of the flow tube, and a second piezoelectric sensor or transducer arranged at a downstream position of the flow tube which, when executed by at least one processor, cause the at least one processor to:
receive a first time-series generated by the ultrasonic flow sensor by causing the first piezoelectric sensor or transducer to transmit at least one first ultrasonic signal to the second piezoelectric sensor or transducer;
receive a second time-series generated by the ultrasonic flow sensor by causing the second piezoelectric sensor or transducer to transmit at least one second ultrasonic signal to the first piezoelectric sensor or transducer, wherein the first time series and the second time series are in a same time interval; and
determine, based on the first time-series and the second time-series, whether to modify a previous amount associated with at least one attribute of the fluid with an interval amount associated with the at least one attribute of the fluid, wherein the interval amount associated with the at least one attribute of the fluid is calculated based on the first time-series and the second-time series.

20. The computer program product of claim 19, wherein the first time series includes a plurality of first amplitudes of the at least one first ultrasonic signal received at the second piezoelectric sensor or transducer at a plurality of first time points in the time interval,
wherein the second time series includes a plurality of second amplitudes of the at least one second ultrasonic signal received at the first piezoelectric sensor or transducer at a plurality of second time points in the time interval, and
wherein whether to modify the previous amount associated with the at least one attribute of the fluid with the interval amount associated with the at least one attribute of the fluid is determined based on at least one of the following: (i) the plurality of first amplitudes and the plurality of second amplitudes, (ii) the plurality of first time points and the plurality of second time points, or any combination thereof.
